(19) United States
(12) Patent Application Publication
(10) Pub. No.: US 2018/0265916 A1
(43) Pub. Date: Sep. 20, 2018

(54) Nucleic Acid Drug Tags and Methods of Tracking the Tags in Bodily Waste

(71) Applicants: David R. Hall, Provo, UT (US); Dan Allen, Springville, UT (US); Conrad Rosenbrock, Provo, UT (US); Andrew Nguyen, Provo, UT (US); Ben Swenson, Lehi, UT (US); Daniel Hendricks, Provo, UT (US)

(72) Inventors: David R. Hall, Provo, UT (US); Dan Allen, Springville, UT (US); Conrad Rosenbrock, Provo, UT (US); Andrew Nguyen, Provo, UT (US); Ben Swenson, Lehi, UT (US); Daniel Hendricks, Provo, UT (US)

(21) Appl. No.: 15/460,575

(22) Filed: Mar. 16, 2017

(51) Int. Cl. C12Q 1/68

(52) U.S. Cl. CPC C12Q 1/6813

(57) ABSTRACT

We disclose a drug tracking system and method of use which may be used to screen a user's bodily waste and to collect information about the drugs the user has consumed. The system includes mixing or adhering a drug tag with a drug compound. The drug tag includes at least one nucleic acid or nucleic acid analog, the unique nucleotide sequence of which correlates to information about the drug. The unique sequence may correlate with a drug molecule, a drug class, a manufacturer, and/or a distributer. Each of these categories of information may be incorporated into the nucleotide sequence of a separate nucleic acid or nucleic acid analog molecules or combined in a single molecule. The sequence of the drug tag may be entered into a database which stores the drug information associated with each discrete nucleotide sequence.

### BACKGROUND

### Field of the Invention

This disclosure relates to methods of tracking and identifying pharmaceuticals after consumption.

### Background of the Invention

The consumption of drugs is largely untracked. This is particularly problematic for dealing with issues such as opiate addiction, sharing of prescriptions, counterfeit drugs, consumption of contraindicated drugs, consumption of multiple drugs with adverse interactions, drug allergies, dosing control and adjustment, emergency medicine and many other situations. It is also difficult to interpret clinical studies when drug compliance of study subjects is inconsistent.

Tracking of drugs in the human waste stream is desirable, though often not practical outside of a clinical setting. Unobtrusive monitoring of the human waste stream is desired. In-toilet monitoring is one potential solution, especially when the toilet has sufficient biometric data on the user to verify identity.

Drug traceability requires a large number of possible categories to track. It may be useful to track the drug molecule, drug category, manufacturer, distributor, physician prescriber, and the dispensing pharmacy. A method of tracking any or all of these in the waste stream is desired.

### BRIEF SUMMARY OF THE INVENTION

We disclose a drug tracking system which includes a drug tracking tag that may be identified in bodily waste. The drug tracking tag includes at least one nucleic acid or nucleic acid analog. The drug tracking tag may be mixed with or adhered to, in the case of a pill, a drug prior to consumption. This step may occur during the manufacturing process.

The nucleic acid or nucleic acid analog may comprise one or a series of unique nucleotide sequences which correlate with specific details about the drug and its supply chain. These may include information in categories that include the identity of the drug, the drug class, the manufacturer, and the distributer. Sequence that includes multiple categories of information may be included in a single nucleotide or nucleotide analog molecule or may be separated into multiple molecules. In some embodiments, the nucleic acid or nucleic acid analog may be conjugated to a magnetic nanoparticle. The magnetic nanoparticle may be less than 8 nm or even less than 5 nm in diameter and may pass through a user's renal system into the urine. The magnetic nanoparticle may be used to isolate the drug tracking tag using magnetic force.

The specific drug tracking tag may be identified in a user's bodily waste by hybridizing the nucleic acid or nucleic acid analog to a complementary single-stranded nucleic acid. The complementary single-stranded nucleic acid may be incorporated into a device which emits a signal upon hybridization and may identify which of a plurality of single-stranded nucleic acids that correlate to codes in drug tracking tags has been hybridized. If necessary, the drug tracking tag may be isolated, amplified, and sequenced.

The information obtained from the nucleic acid or nucleic acid analog within the drug tracking tag in the form of nucleotide sequence may be entered into a database which stores all of the sequence codes used in drug tracking tags and their corresponding information about the drug. By relating the sequence to the drug information associated with it, the drug compound the user consumed and its drug delivery chain may be determined. The information may also be compared to the user's prescribed medications to assess drug compliance and with known drug interactions to prevent adverse events.

### BRIEF DESCRIPTION OF THE DRAWINGS

FIG. 1 illustrates a schematic drawing of a nucleotide or nucleotide analog which includes a sequence that encodes for a variety of types of information about a drug.

FIG. 2 illustrates a schematic drawing of a plurality of nucleotides or nucleotide analogs which each include a sequence that encodes information of a single category.

FIG. 3 illustrates a schematic drawing of a nucleotide or nucleotide analog that is conjugated to a magnetic nanoparticle.

FIG. 4A illustrates a schematic drawing of a microarray system which may be used to hybridize to nucleic acids or nucleic acid analogs to identify the presence of the nucleic acid or nucleic acid analog.

FIG. 4B illustrates the microarray system of FIG. 4A which has hybridized to a nucleic acid.

FIG. 5 illustrates a table which correlates nucleotide sequences with drug classes.

FIG. 6 illustrates a flow chart which includes steps in an embodiment of a method of using the disclosed drug tracking system.

FIG. 7 illustrates a flow chart including steps for using the disclosed drug tracking system to confirm user drug compliance.

FIG. 8 illustrates a flow chart including steps for using the disclosed drug tracking system to identify drug interactions.

### DETAILED DESCRIPTION OF THE INVENTION

Definitions

Drug, as used herein, means any pharmacologically active agent or mixture of agents.

Drug consumption, as used herein, means taking a drug into the body through any method of administration.

Nucleotide, as used herein, means a molecule which is a monomer used to synthesize polymeric nucleic acids and nucleic acid analogs. A nucleotide, as used herein, comprises three subunit molecules: a nitrogenous base, which may be an unnatural base, a five-carbon sugar, and at least one phosphate group.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a drug tracking system which includes a drug tag that is capable of tracking multiple points of origin in the drug delivery chain and of providing multiple types of information about the drug itself. This drug tag may be detected in bodily waste. The drug tag may include at least one nucleic acid or nucleic acid analog that has a unique nucleotide sequence. The unique sequence may correspond to one or any combination of the following classifications: a drug molecule, a drug class, a drug manufacturer, and a drug distributor. Drug classes which may be represented by a unique nucleotide sequence within the drug tag include, but are not limited to: opioids, antibiotics, antihypertensive compounds, steroid hormones, antihistamines, antidepressant compounds, antipsychotic compounds, antiviral compounds, antifungal compounds, anti-inflammatory compounds, anticonvulsants, diuretics, muscle relaxants, statins, and immunosuppressive agents.

In some embodiments, the nucleic acid or nucleic acid analog may comprise between approximately 80 and 100 base pairs. In other embodiments, the nucleic acid or nucleic acid analog may comprise between approximately 30 and 50 base pairs. In other embodiments, the nucleic acid or nucleic acid analog may comprise between approximately 4 and 10 base pairs. In some embodiments, the nucleic acid or nucleic acid analog may be either deoxyribonucleic acid or ribonucleic acid. In some embodiments, the nucleic acid or nucleic acid analog may be either a double- or single-stranded molecule.

In some embodiments, a single nucleic acid or nucleic acid analog may contain a set of sequences corresponding to each of the classifications listed. This may be desirable, for example, in cases where a user is consuming multiple drugs. Including all the drug information on the same molecule within the drug tag would make it clear which of the multiple pieces of information on the drug tag refer to a particular drug product.

In other embodiments, nucleotide sequences corresponding to each of the classifications of drug molecule, drug class, drug manufacturer, and drug distributor may each be on separate nucleic acids or nucleic acid analogs. This may be desirable in cases where the drug manufacturer sends a drug to many distributers. In such cases, a manufacturer may insert the nucleic acids or nucleic acid analogs with the nucleotide sequences that correspond to the drug molecule, class, and manufacturer into the drug. Later, the distributor may add a nucleic acid or nucleic acid analog with the nucleotide sequence corresponding to the distributor. This option may simplify the addition of drug tags to drugs by manufacturers who produce many drug molecules or drug classes.

The drug tag may be added to the drug through one or more of the following methods: spraying, dipping, painting, mixing with drug constituents, or mixing with the material from which a capsule or gelcap is formed. Other methods known in the art may also be used to apply the drug tag to the drug product.

To identify the drug tag after it has been excreted, it may be useful to extract the drug tag from the bodily waste. In some embodiments, the nucleic acid or nucleic acid analog may be tethered to a magnetic nanoparticle. The magnetic nanoparticle may provide a method of extracting the drug tag from bodily waste using magnetic force. The magnetic nanoparticle may be between approximately 5 and 10 nm in diameter. In other embodiments, the magnetic nanoparticle may be between approximately 3 and 8 nm in diameter.

In any embodiment that includes a magnetic nanoparticle, the magnetic nanoparticles may be small enough to pass through the renal system such that they may be excreted in the urine stream. The magnetic nanoparticles may then be separated from the urine with a high magnetic field gradient region, for example, a microneedle array.

In one example of method of synthesizing an embodiment of the disclosed drug tag that includes a magnetic nanoparticle, the magnetic nanoparticle is conjugated to a nucleotide or nucleotide analog in solution with a surplus of magnetic nanoparticles so as to have a small chance of more than one nucleotide or nucleotide analog conjugating to a magnetic nanoparticle. This technique may leave many magnetic particles with no nucleotide or nucleotide analog. Consequently, the drug tag may not include magnetic nanoparticles with more than one nucleotide or nucleotide analog, a situation which would increase the size of the drug tag. In addition, the nucleotide or nucleotide analog may not coil around the nanoparticle, which could increase the diameter of the nanoparticle.

In some embodiments, a method of detecting a drug in bodily waste may be described by the following steps. First, a sample of bodily waste may be obtained from a user who has consumed a drug compound that includes a drug tag. In some cases, a diagnostic toilet that is equipped with the appropriate equipment may be used to collect and analyze the urine as described. This may be particularly desirable when the diagnostic toilet already has sufficient biometric data to identify the user. Next, the identity of the unique sequence within the nucleic acid or nucleic acid analog that includes information about the drug may be determined. This may be accomplished using microarray technology. Alternatively, other techniques which includes single-stranded nucleic acids that are homologous to the unique sequence within the drug tag may be used to identify the drug information within the tag. These techniques may include devices which emit a signal when a nucleic acid or nucleic acid analog from the drug tag hybridizes to the homologous nucleic acid. Examples include a complementary metal oxide semiconductor (CMOS) biosensor chip using self-sensing piezoresistive microcantilevers as the molecules hybridize as described in U.S. Pat. No. 7,738,086 which is hereby incorporated by reference in its entirety. Alternatively, the detection device may include technology described by Star et al. ((2005) Proc. Natl. Acad. Sci. USA, 103, 921-926) which is hereby incorporated by reference in its entirety. Star et al describe a technique using carbon nanotube network field-effect transistors (NTNFETs) that function as selective detectors of DNA immobilization and hybridization. Finally, the nucleotide sequence which hybridized the homologous nucleic acid may be identified such that it can be located within a database. In some embodiments, additional steps may include amplifying the nucleic acid or nucleic acid analog prior to identification and/or quantifying the amount of nucleic acid or nucleic acid analog present in the sample of bodily waste. The nucleic acid or nucleic acid analog may also be sequenced using technology known in the art.

In embodiments of the drug tag that include a magnetic nanoparticle, the drug tag may be removed from bodily waste, which in some embodiments may occur with the use of an array of magnetic microneedles in a flow cell. The nucleic acid or nucleic acid analog may then optionally be cleaved from the magnetic nanoparticle for further analysis. This may be accomplished through enzyme action, thermal action, chemical action, photoaction, or any other methods of removing nucleic acids or nucleic acid analogs known in the art.

The system of tracking and identifying drug tags may also include a database which contains entries that associate the specific nucleotide sequence of the nucleic acid or nucleic acid analog to the drug molecule, drug class, drug manufacturer, drug distributor, or combination of these to which the sequence corresponds. Following the identification of the nucleotide sequence or sequences present in a sample of bodily waste, the nucleotide sequence or sequences may be compared to the entries of nucleotide sequences in the database, such that the drug molecule, class, manufacturer, and/or distributor to which the nucleotide corresponds may be identified.

If a user has consumed more than one drug, it may be desirable to cross-check the drugs consumed with the user's prescription to identify a potentially harmful combination of drugs. Thus, in some embodiments of the invention, the step of identifying the classifications of drug molecule, class, manufacturer, and distributor for any drug in a sample may be followed by a comparison of the drugs in the sample to the user's prescriptions. The identity of the drug may be entered into the database which also includes a file that lists the drugs which have been prescribed to the user. This allows the drug tracking system to confirm that the user is consuming the correct medication and being compliant with the prescription specifications.

The drug tracking system may also be used to identify drug interactions that may cause an adverse effect. For example, after two or more drugs have been identified using the drug tracking system, the two or more drug molecules may be compared to additional entries in the database which may include information about drug interactions. An adverse event may thereby be avoided.

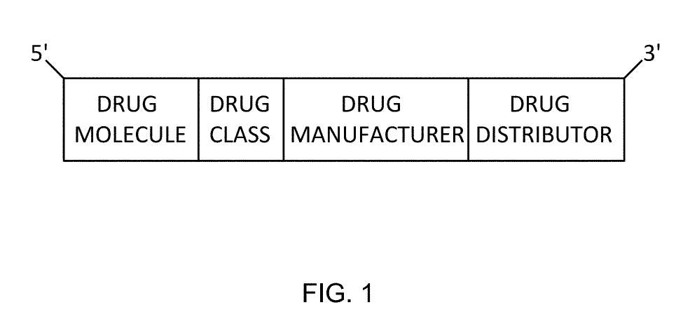

Referring now to the drawings, FIG. 1 shows a schematic drawing of an embodiment of a nucleotide or nucleotide analog according to the disclosed drug tracking system. The nucleotide or nucleotide analog of FIG. 1 includes four discreet sections of nucleotides. The nucleotide sequence of each of the discreet sections includes coded information about the drug with which the drug tracking tag is associated. The first section includes a code that identifies the specific drug molecule, the second section identifies the drug class, the third section identifies the drug manufacturer, and the fourth identifies the drug distributer. In this embodiment, all of the information, in the form of unique nucleotide sequences, is included in one molecule.

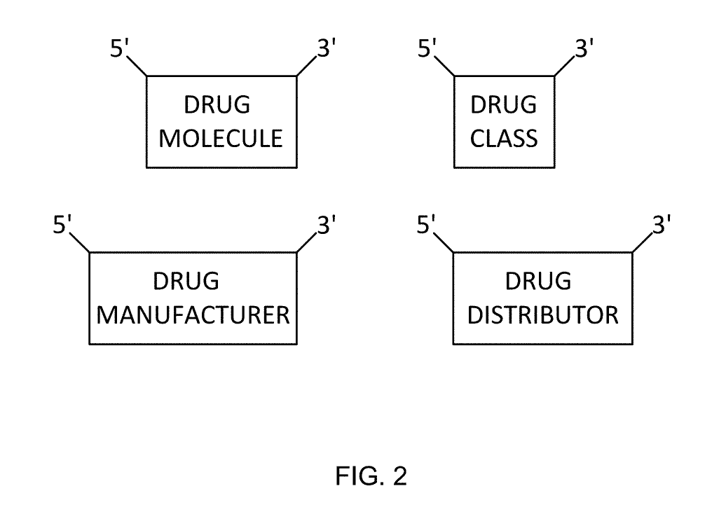

FIG. 2 shows a schematic drawing of an embodiment of multiple nucleotides or nucleotide analogs according to the disclosed drug tracking system. Each category of information about the drug is included in a separate molecule. The categories include the identity of the drug molecule, the drug class, the drug manufacturer, and the drug distributer. Like the embodiment of FIG. 1, each molecule shown in FIG. 2 encodes the information using unique nucleotide sequences.

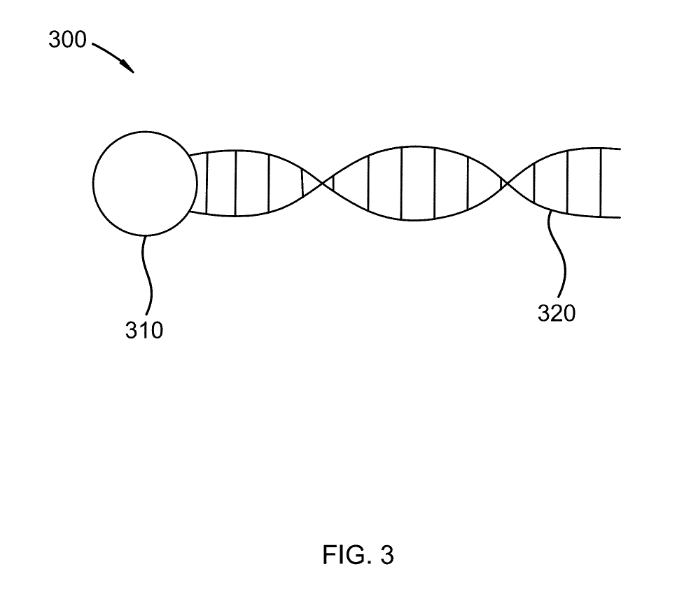

FIG. 3 illustrates drug tag 300. Drug tag 300 is an embodiment of a nucleotide or nucleotide analog according to the disclosed drug tracking system which is conjugated to magnetic nanoparticle 310. Magnetic nanoparticle 310 is conjugated to nucleotide 320. Nucleotide 320 may include information about the drug with which drug tag 300 may be associated. The information may include identity of the drug molecule, the drug class, the drug manufacturer, and the drug distributer. Magnetic nanoparticle 310 may be used to isolate drug tracking tag 300 from a sample of biological waste.

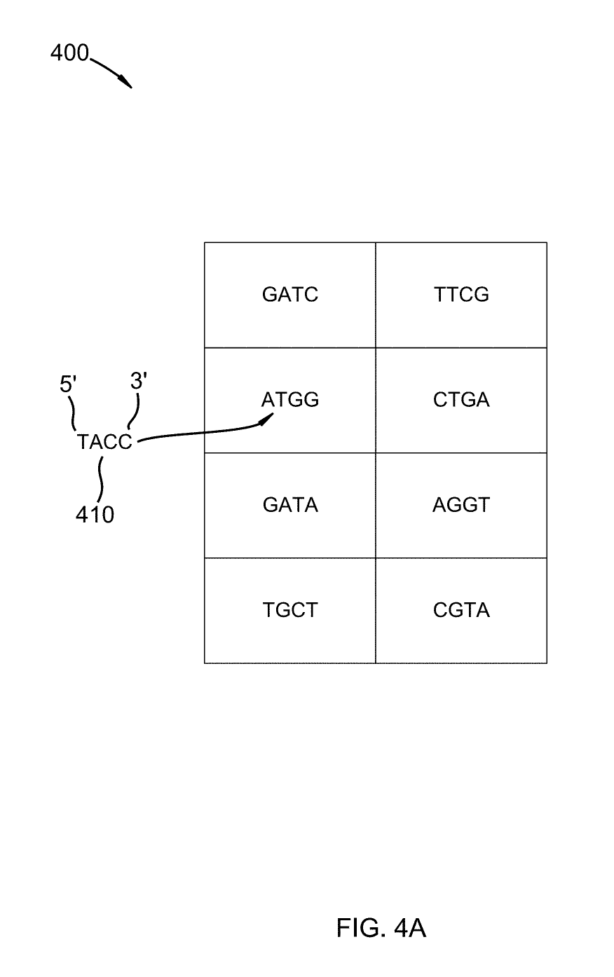

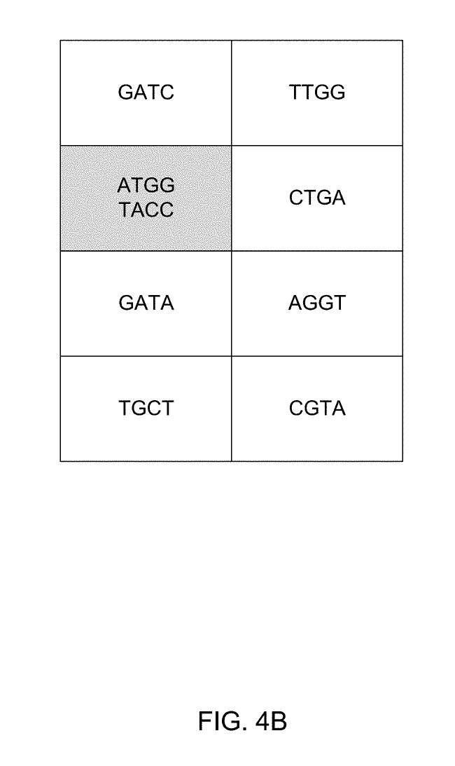

FIG. 4A is a schematic illustration of a means for identifying the presence of a nucleotide or nucleotide analog which may be part of a drug tag according to an embodiment of the disclosure. FIG. 4A illustrates an array, which may be a microarray, with a plurality of single-stranded nucleic acid molecules adhered to each section of the array. Each of the single-stranded nucleic acid molecules has a unique sequence that is complementary to one of the unique sequences used in the drug tags. A drug tag that includes the sequence that is complementary to one of the single-stranded nucleotides on the array will hybridize only with its complementary sequence.

FIG. 4B illustrates the array of FIG. 4A after a complementary sequence from a drug tag has hybridized to a single-stranded nucleic acid on the array. In some embodiments, the array may emit a signal when the hybridization occurs. The signal may further indicate which of the single-stranded nucleic acids on the array has been hybridized.

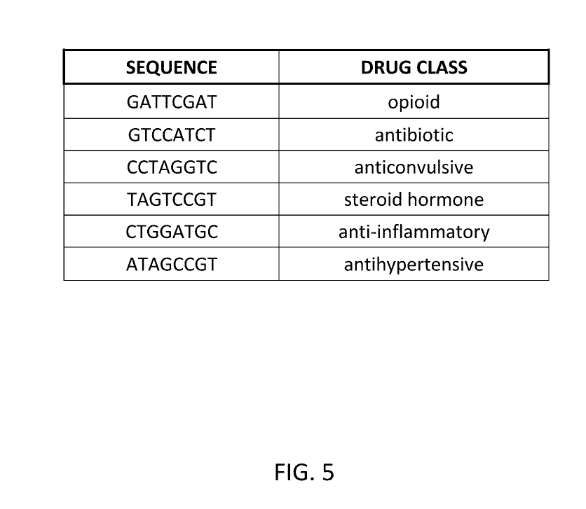

FIG. 5 shows a table which illustrates the association between unique nucleotide sequences which may be present on a nucleic acid or nucleic acid analog which is part of a drug tag according to the disclosure.

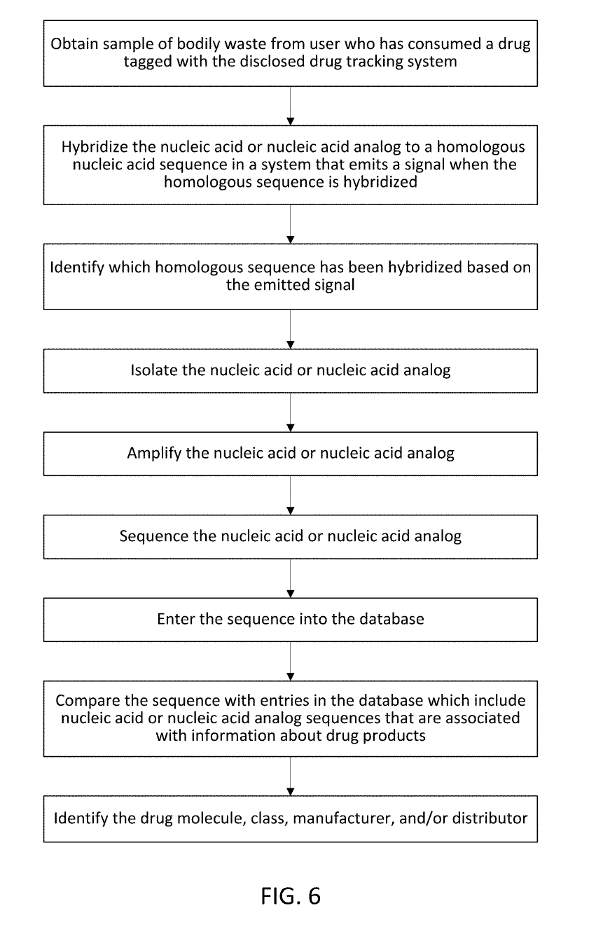

FIG. 6 provides a flow chart which outlines steps which may be used to use the disclosed drug tracking system to obtain information about a drug a user has consumed. The steps include obtaining a sample of bodily waste from a user. This may be accomplished when the user deposits bodily waste into a toilet that is equipped to detect the drug tags. Next, the nucleic acid or nucleic acid analog on the drug tag may be hybridized to a homologous nucleic acid sequence in a system that emits a signal with the homologous sequence is hybridized. This system may be that described in FIGS. 4A and 4B and may be incorporated into a toilet. The specific homologous nucleic acid sequence which hybridized with the nucleic acid or nucleic acid analog of the drug tag is then identified. In some situations, this last step may provide all the information that is needed. However, the drug tracking tag may include coded information other than that associated with the part of the molecule that hybridized with the array. In fact, the particular sequence with which the drug tag hybridized may prompt the need for additional information. For example, the hybridization assay may indicate that the user has consumed an opioid. When substance abuse is suspected, more detailed information about the drug the user consumed may be needed. Therefore, the drug tag may be isolated and the nucleic acid or nucleic acid analog may be amplified. The sequence of the amplified nucleic acid or nucleic acid analog may be determined using sequencing techniques known in the art.

Regardless of whether the drug tag was amplified and/or sequenced, the sequence may be entered into a database and compared to a plurality of sequences stored in the database which are associated with unique pieces of information about the drug associated with the drug tag. By associating the sequence of the drug tag with its associated data, information about the drug the user consumed may be obtained. This may include the identity of the drug molecule, the drug class, the manufacturer, and/or the distributor.

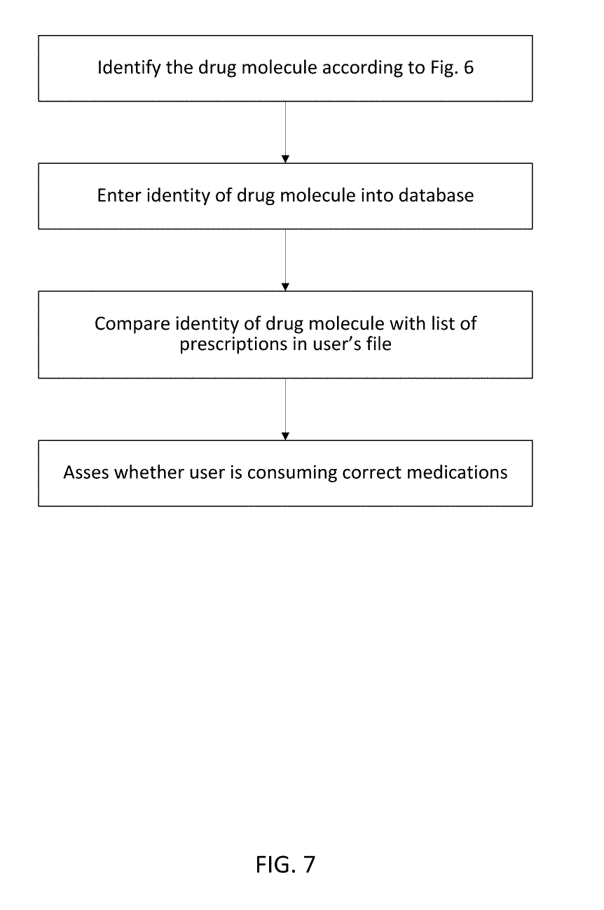

FIG. 7 is a flow chart illustrating how the disclosed drug tag may be used to confirm user drug compliance. In this method, the identity of the drug the user consumed is first determined using the method of FIG. 6. The identity of the drug is then entered into a database that includes a file listing the drugs that have been prescribed to the user. The identity of the drug is compared to the list of one or more drugs prescribed for the user. This comparison may identify whether the user has consumed the proper drug compounds as prescribed.

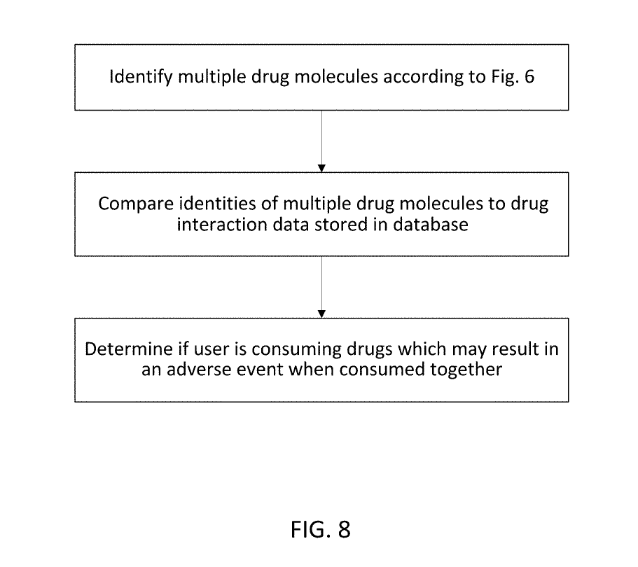

FIG. 8 is a flow chart illustrating how the disclosed drug tracking tag may be used to assess whether a user has consumed multiple drugs which, when taken together, may result in an adverse reaction. The steps include identifying the drugs the user has consumed using the method of FIG. 6. In this embodiment, multiple drugs may be identified by screening the user's bodily waste for drug tags. The identities of the drugs the user has consumed may be entered into a database that has stored information about a plurality of drugs which cause adverse events when consumed together. The drugs identified as those the user has consumed are cross-checked with the drug interaction information in the database to determine if the drugs the user has consumed may cause an adverse event.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A drug tracking system comprising:
a drug tag, the drug tag comprising at least one nucleic acid or nucleic acid analog, wherein the at least one nucleic acid or nucleic acid analog comprises a unique nucleotide sequence that correlates with at least one of the following: a drug molecule, a drug class, a drug manufacturer, and a drug distributor;
a database, the database comprising entries which associate nucleotide sequences of nucleic acids or nucleic acid analogs with each of the following: a drug molecule, a drug class, a drug manufacturer, and a drug distributor to which each sequence corresponds.

2. The drug tracking system of claim 1, wherein the nucleotide sequences corresponding to the drug molecule, the drug class, the drug manufacturer, and the drug distributor are each present on a single nucleic acid or nucleic acid analog.

3. The drug tracking system of claim 1, wherein the nucleotide sequences corresponding to the drug molecule, the drug class, the drug manufacturer, and the drug distributor are each present on a separate nucleic acid or nucleic acid analog.

4. The drug tracking system of claim 1, wherein the nucleic acid or nucleic acid analog comprises between about 80 and about 100 base pairs.

5. The drug tracking system of claim 1, wherein the nucleic acid or nucleic acid analog comprises between about 30 and about 50 base pairs.

6. The drug tracking system of claim 1, wherein the nucleic acid or nucleic acid analog comprises between about 4 and about 10 base pairs.

7. The drug tracking system of claim 1, wherein the nucleic acid or nucleic acid analog comprises deoxyribonucleic acid.

8. The drug tracking system of claim 1, wherein the nucleic acid or nucleic acid analog consists of a double-stranded molecule.

9. The drug tracking system of claim 1, wherein the nucleic acid or nucleic acid analog consists of a single-stranded molecule.

10. The drug tracking system of claim 1, wherein the at least one nucleic acid or nucleic acid analog is conjugated to a magnetic nanoparticle.

11. The drug tracking system of claim 10, wherein the diameter of the magnetic nanoparticles is between about 5 nm and about 10 nm.

12. The drug tracking system of claim 10, wherein the diameter of the magnetic nanoparticles is between about 3 nm and about 8 nm.

13. A method of detecting a drug in bodily waste comprising the steps of:
obtaining a sample of bodily waste from a user who has consumed a drug molecule, wherein the drug molecule is connected to a drug tag according to claim 1; and
hybridizing the nucleic acid or nucleic acid analog of the drug tag to a single-stranded nucleic acid, wherein the single-stranded nucleic acid comprises a sequence which is homologous to the nucleic acid or nucleic acid analog of the drug tag, and wherein the single-stranded nucleic acid is connected to a means for emitting a signal when the single-stranded nucleic acid hybridizes to the nucleic acid or nucleic acid analog of the drug tag.

14. The method of claim 13, further comprising the step of amplifying the at least one nucleic acid or nucleic acid analog of the drug tag.

15. The method of claim 13, further comprising the step of quantifying the at least one nucleic acid or nucleic acid analog of the drug tag.

16. The method of claim 13, further comprising the step of identifying the sequence of the at least one nucleic acid or nucleic acid analog of the drug tag.

17. The method of claim 16, further comprising the steps of:
entering the nucleotide sequence of the at least one nucleic acid or nucleic acid analog of the drug tag into the database of claim 1;
comparing the nucleotide sequence of the at least one nucleic acid or nucleic acid analog of the drug tag to a plurality of nucleotide sequence entries in the database of claim 1, wherein the nucleotide sequence entries are associated with one or more of the following: a first drug molecule, a first drug class, a first drug manufacturer, and a first drug distributor to which each sequence corresponds; and
identifying the first drug molecule, the first drug class, the first drug manufacturer, or first drug distributor associated with the nucleotide sequence of the at least one nucleic acid or nucleic acid analog of the drug tag.

18. The method of claim 17, wherein the drug class which correlates to the at least one nucleic acid or nucleic acid analog sequence of the drug tag consists one or more of the following: opioids, antibiotics, antihypertensive compounds, steroid hormones, antihistamines, antidepressant compounds, antipsychotic compounds, antiviral compounds, antifungal compounds, anti-inflammatory compounds, anticonvulsants, diuretics, muscle relaxants, statins, and immunosuppressive agents.

19. The method of claim 17, further comprising the step of comparing the first drug molecule, the first drug class, the first drug manufacturer, or first drug distributor with a user's file which comprises a list of drugs prescribed to the user.

20. The method of claim 17, further comprising the steps of:
identifying the sequence of a second nucleic acid or nucleic acid analog of a second drug tag wherein the second drug tag is according to claim 1;
entering the sequence of the second nucleic acid or nucleic acid analog of the second drug tag into the database of claim 1; and
comparing the sequence of the second nucleic acid or nucleic acid analog to a plurality of sequence entries in the database of claim 1 which are associated with a plurality of drug molecules;
identifying a second drug molecule;
comparing the identities of the first drug molecule and second drug molecule with a plurality of entries in the database which include drug interactions.
